(19) United States
(12) Patent Application Publication
(10) Pub. No.: US 2005/0112196 A1
(43) Pub. Date: May 26, 2005

(54) Rapidly disintegrating formulation

(76) Inventors: Jianbo Xie, Davie, FL (US); Guohua Zhang, Davie, FL (US); Xiu Xiu Cheng, Weston, FL (US); Roger Carter, Miami, FL (US)

Correspondence Address: Martin P. Endres, Esq.;HEDMAN & COSTIGAN, P.C., 1185 Avenue of the Americas, New York, NY, 10036, US

(21) Appl. No.: 10/959,902

(22) Filed: Oct. 6, 2004

Related U.S. Application Data
(60) Provisional application No. 60/509,327, filed on Oct. 7, 2003.

(51) Int. Cl. A61K 31/551; A61K 31/519; A61K 9/20

(52) U.S. Cl. 424/464; 514/220; 514/259.41

(57) ABSTRACT

The invention relates to a rapidly disintegrating oral dosage formulation that contains a water insoluble or slightly water soluble neurological agent and method of preparing the rapidly disintegrating formulation wherein the formulation is designed to dissolve in the buccal cavity of the patient.

This application claims the benefits of provisional application 60/509,327 filed Oct. 7, 2003.

### FIELD OF THE INVENTION

The present invention relates to the field of oral dosage forms and in particular the field of rapidly disintegrating oral dosage formulations which disintegrate rapidly in the saliva of the buccal cavity and can be swallowed easily with or without drinking water. As used in this application the term “rapidly disintegrating” means that the dosage formulation dissolves in an aqueous media within 5 minutes, preferably less than two minutes and most preferably less than one minute. In an embodiment of the present invention the drug or active pharmaceutical ingredient in the dosage form is a slightly soluble to water insoluble neurological agent such as a neuroleptic or psychopharmacologic agent.

### BACKGROUND OF THE INVENTION

Rapid orally disintegrating dosage formulations are known in the art. Some rapidly disintegrating dosage formulations are described in U.S. Pat. Nos. 4,371,516; 5,178,878; 5,298,261; 5,464,632, 5,587,180; 5,720,974; 5,807,576; 5,866,163; 5,869,098, 6,024,981, 6,048,541, 6,149,938 and 6,316,029 which are incorporated herein by reference. The prior art rapidly disintegrating formulations frequently require complicated processing techniques such as lyophilization, foam techniques or specialized excipients such as effervescents, highly micronized agents or the like. These prior art rapidly disintegrating tablets are generally large tablets in excess of 500 mg or more in total weight and often result in some discomfort in the mouth due to size.

Among some of the aforementioned prior aft formulations is the one described in U.S. Pat. No. 5,178,878 to Wehling et al., which discloses a rapidly dissolving oral formulation that requires an extragranular microparticulate active in conjunction with an effervescent agent incorporated into a tableted matrix in order to achieve its rapid oral disintegration. Examples therein result in tablets with a total weight greater than 500 mg. U.S. Pat. No. 6,024,981 to Khankari et al., discloses a rapid oral disintegrating tablet that minimally requires a matrix composed of a lubricant and a non-direct compression filler, which as the reference discloses, imparts an advantage over direct compression filler such as commercial mannitol having a minimum of at least about 80% average particle size over 100 microns. Examples therein result in total tablet weight well in excess of 500 mg.

Some commercially available rapidly disintegrating tablets that contain either water insoluble or slightly water soluble neurological agents are ZYPREXA® ZYDIS® which is a rapidly disintegrating tablet that contains the drug olanzapine and preservatives sodium methyl paraben and sodium propyl parabin; RISPERDAL® M-TAB which is a rapidly disintegrating tablet that contains the drug risperidone and a carrier resin, AMBERLITE®; REMERON SOLTAB® which is a rapidly disintegrating tablet that contains the drug mirtazapihe and an effervescent agent, sodium bicarbonate; and, PARACOPA® which is a rapid orally disintegrating tablet containing the therapeutic combination of carbidopa and levodopa. Another example of a commercially available rapid orally dissolving formulation include the well-known CLARITIN® REDITABS which contains the drug loratadine.

It is an objective of the present invention to provide a safe and effective rapidly disintegrating oral dosage formulation that can be economically be prepared.

It is a further object of the present invention to provide a rapidly disintegrating oral dosage formulation that weighs less than 500 mg, preferably less than 400 mg and most preferably less than 300 mg.

It is an additional object of the present invention to provide a rapidly disintegrating oral dosage formulation for active pharmaceutical ingredients that are slightly soluble or insoluble in water.

It is still a further object of the present invention to provide a rapidly disintegrating oral dosage form containing neurological agents such as neuroleptics, psychotropic agents and antidepressant agents.

It is also an object of the present invention to provide a rapidly disintegrating oral dosage formulation that can be manufactured by direct compression without the need for special manufacturing techniques such as lyophilization or special excipients such as charged resins, preservatives or effervescent agents.

### SUMMARY OF THE INVENTION

The forgoing objectives and others are met by the present invention. The present invention is a rapid orally disintegrating pharmaceutical solid dosage form for water insoluble or slightly soluble pharmaceutically active ingredients. Active pharmaceutical ingredient includes one or more chemical compounds (i.e. drugs) having a therapeutic effect on a patient. A rapid orally disintegrating solid pharmaceutical dosage form can be typically defined in the art as a solid that dissolves when contacting saliva and any other fluids present in the oral cavity of the patient as further described below. Some preferred water insoluble or slightly water soluble pharmaceutical ingredients are neurological agents that include the neuroleptics and the group of psychopharmacological agents known as psychotropics such as antipsychotics and antidepressants that can be compressed into a tablet using conventional pharmaceutical tableting techniques to yield a total tablet weight tablet less than 500 mg, preferably less than 400 mg and most preferably less than 300 mg.

The water insoluble or slightly soluble pharmaceutically active ingredient is combined with conventional pharmaceutical excipients such as fillers, preferably directly compressible fillers, binders, taste enhancing agents, disintegrants and stabilizers then compressed into a tablet using conventional pharmaceutical tableting techniques. Preferably the active is granulated in the presence of a polymer wherein the weight percentage of the polymer relative to the total weight of the granulate is less than 30. In a more preferred embodiment, the polymer will be selected from any of the water soluble or water dispersible polymers well-known in the art. The total weight of the final tablet of the present invention is less than 500 mg, preferably less that 400 mg and most preferably less than 300 mg.

The rapidly disintegrating oral dosage formulation of the present invention may also contain conventional processing aids such as solubilizers, glidants, lubricants, dyes and pigments. These conventional processing aids are well know to the skilled artisan and are used in amounts that do not materially affect the final properties of the dosage formulation.

The rapidly disintegrating oral dosage formulation of the present invention can be prepared by any of the conventional processing techniques known in the art, however, the preferred method involves granulation with the active and the subsequent tableting of the granules. The most preferred method involves: a) preparing a wet granulation of the drug, a binder, preferably a water soluble polymeric binder, a directly compressible filler, a taste enhancing agent, a distintegrant and optionally a stabilizer; b) blending the granules from step (a) with additional filler, taste enhancing agent, disintegrants and optionally a stabilizer; and c) compressing the blend of step (b) into a tablet. Preferably the binders, fillers and disintegrants used in the present invention are all water soluble to reduce the unpleasant grittiness sometimes associated with the use of water insoluble materials. Also if mannitol is used in the present invention, it is preferred that the total amount of mannitol be less than 50 weight percent of the total tablet weight and most preferably no mannitol is used in the granules.

### DETAILED DESCRIPTION OF THE INVENTION

In its more preferred embodiments, the rapid orally disintegrating solid dosage formulation of the present invention may comprise the following ranges of ingredients:

| INGREDIENT | PREFERRED | MOST PREFERRED |
|---|---|---|
| ACTIVE | 0.1-20% | 0.25-10% |
| FILLER | 40-95% | 60-90% |
| BINDER | 0.5-20% | 1.0-10% |
| TASTE ENHANCING AGENT | 0.5-15% | 1.0-10% |
| DISINTEGRANT | 0.5-20% | 1.0-15% |
| STABILIZER | 0-15% | 0.5-10% |

All the percentages in the above table are based on the total weight of the final dosage formulation.

In an alternate embodiment the present invention will comprise a mixture of granules and tabletting excipients. The granules will comprise the following ingredients:

| INGREDIENT | PREFERRED | MOST PREFERRED |
|---|---|---|
| ACTIVE | 1.0-45% | 2.5-35% |
| FILLER | 30-80% | 40-75% |
| BINDER | 0.1-30% | 0.5-25% |
| TASTE ENHANCING AGENT | 0.5-20% | 1.0-15% |
| DISINTEGRANT | 0.1-15% | 0.5-10% |
| STABILIZER | 0-25% | 1.0-15% |

The percentages in the above table are based upon the total weight of the granules.

The tabletting excipients will comprise the following ingredients:

| INGREDIENT | PREFERRED | MOST PREFERRED |
|---|---|---|
| FILLER | 50-98% | 65-95% |
| BINDER | 0.1-20% | 0.5-15% |
| TASTE ENHANCING AGENT | 0.5-15% | 1.0-10% |
| DISINTEGRANT | 0.5-25% | 1.0-20% |
| STABILIZER | 0-15% | 0.5-10% |
| LUBRICANT/GLIDANT | 0.25-10% | 0.5-5% |

The percentages in the above table are based upon the total weight of the tableting excipients.

As used herein the term “slightly soluble” means from 100 to 1000 parts of water are required to dissolve 1 part of the drug and the term “insoluble” means greater than 1000 parts of water are required to dissolve 1 part of the drug or less.

Preferably the water insoluble or slightly soluble drugs are neurological agents that include neuroleptics and psychopharmacological agents such as antipsychotic drugs and antidepressant drugs. Some common psychopharmacological agents are described in Remington, The Science and Practice of Pharmacy 20^{th} ed. and are incorporated herein by reference. Psychotropic agents may include: antianxiety, antidepressant, antimanic, antipanic, antipsychotic, or phenothiazines, or combinations thereof. Some examples of antipsychotic drugs useful in the present invention are fluphenazine, decanoate, haloperidol, loxapine succinate, thiothixene, clozapine, olanzapine and risperidone. Some examples of antidepressant drugs useful in the present invention are amoxapine, fluvoxamine maleate, imipramine pamoate, mirtazapine, trazodone hydrochloride and trimipramine maleate. Examples of neuroleptics suitable for the present invention include decarboxylase inhibitors such as carbidopa and levodopa as well as catechol methyltransferase inhibitors such as entacapone. Moreover, the present invention would, as expected, include all pharmaceutically acceptable salts, isomers, metabolites and polymorphic forms of the foregoing agents provided they are slightly soluble to insoluble in water.

The filler used in the formulation may be any pharmaceutically acceptable filler or diluent. Some of the preferred fillers are lactose, starch, dextrose, sucrose, fructose, maltose, mannitol, sorbitol, kaolin, microcrystalline cellulose, powdered cellulose or any combination of the foregoing. In a preferred embodiment of the present invention, the filler consists of a mixture of water soluble fillers to reduce the chance of unpleasant grittiness when the tablet dissolves in the oral cavity of the patient. Most preferably, the filler will be a direct compression sugar such as confectioners sugar, dextrates, dextrin, dextrose, fructose, maltose, mannitol, polydextrose, sorbitol, or other sugars and sugar derivatives.

The binder may be any pharmaceutically acceptable binder. The binder is preferably a water soluble polymer of the group consisting of polyvinyl alcohol, polyvinylpyrrolidone, methylcellulose, hydroxypropyl cellulose, hydroxymethyl cellulose and any combination of the foregoing. Polyvinylpyrrolidone is the most preferred binder.

The disintegrant used in the present invention can be selected from the group consisting of corn starch, croscarnelose sodium, crospovidone (polyplasdone XL-10), sodium starch glycolate (EXPLOTAB or PRIMOJEL) or any combination of the foregoing. The most preferred disintegrant is crospovidone or sodium starch glycolate.

The flavoring agents preferably are taste enhancing agents and can include artificial sweeteners such as aspartame, saccharin, dipotassium glycyrrhizinate, stevia, thaumatin and flavorants such as citric acid, peppermint oil, wintergreen oil, menthol, lemon, lime, orange grape, cherry and vanilla extract. Additional taste enhancing agents are described in U.S. Pat. No. 6,027,746 and are incorporated herein by reference. In a preferred embodiment of the present invention, the flavoring agent is preferably a taste enhancing agent and may comprise a mixture of artificial sweeteners and flavorants such as aspartame and peppermint oil or grape extract.

The stabilizers used in the present invention can be any stabilizer commonly known in the industry and the selection will depend upon the properties of the drug employed in the dosage formulation. For example, if the drug is sensitive to basic environments, an acidic stabilizer should be used such as citric, fumaric or tartaric acid. Similarly if the drug is sensitive to acidic environments, a basic stabilizer should be used such as sodium dihydrogen phosphate, calcium or magnesium carbonate, arginine, lysine or meglamine. A list of possible stabilizers can be found in the Handbook of Pharmaceutical Excipients and U.S. Pat. No. 6,316,029 which are incorporated herein by reference. The present invention may also comprise conventional processing aids such as tablet lubricants (magnesium stearate, sodium stearate), glidants (colloidal silicon dioxide) and wetting agents or solubilizers (sodium lauryl sulfate, polysorbates). The processing aids are generally added to the dosage formulation in small amounts (less than 5 weight percent of the total weight of the formulation) and do not materially affect the properties of the final dosage formulation. Some of the aforementioned excipients can perform more than one function in the formulation. For example, glyceryl behenate and sodium stearyl fumarate can function as both a lubricant and a stabilizer. The multi-function excipients are known to those skilled in the art.

The following examples illustrates the present invention and is not intended to limit the scope of the present invention.

### EXAMPLE 1

An antipsychotic tablet containing risperidone is prepared according to the following procedure:

Stage I: Granulation

16 kg of risperidone granules were prepared by placing 14.96 kg of ethyl alcohol SDA 3a 190 proof (ethanol) in a stainless steel container equipped with a mechanical stirrer. 0.159 kg of peppermint oil was added to the ethyl alcohol and stirred for approximately 5 minutes. 2.672 kg of purified water was then added to the ethyl alcohol and peppermint oil followed 1.618 kg of L-Tartaric acid NF. The mixture was stirred for and additional ten minutes. While stirring, 0.8006 kg of risperidone was then added to the mixture and stirred for and additional ten minutes. 2.418 kg of povidone USP (Kollidon K-30) was then added to the mixture and stirred until the povidone was completely dissolved, approximately 30 minutes.

1.618 kg of aspartame, 0.098 kg of colloidal silicon dioxide, NF (CAB-O-SIL M-5), 0.338 kg of crospovidone, NF (polyplassdone XL-10), 11.597 kg of Dextrates NF hydrated (EMDEX) were charged into a GPCG 15 Glatt fluidized bed coater. The risperidone mixture prepared above was then sprayed onto the contents of the fluidized bed coater using the following target parameters:

| | |
|---|---|
| Spray position: | top spray |
| Insert size: | 45 L |
| Filter: | 2.5 microns |
| Screen Size: | 200 mesh |
| Nozzle Tip Diameter: | 1.5 mm |
| Filter Bag Shake Cycle: | 3 sec. every 30 sec. |
| Inlet Air Volume: | 400 SCFM (200-600 SCFM) |
| Atomization Pressure: | 3.0 bar (2.0-4.0 bar) |
| Spray Rate: | 100-400 mL/min |
| Product Temperature: | 35° C. (25°-55° C.) |
| Tubing Size: | 24 mm |

Once the risperidone mixture was consumed, the resulting granules were dried in the fluidized bed until the loss on drying was less than 5%. The dried granules were removed from the fluidized bed and screen using a Comil equipped with a # 1143 screen and spacer. The screened granules were then placed in a 2 cu. ft. V-Blender and blended at the maximum speed for about 7 minutes.

Stage II: Tableting

Risperidone tablets containing 0.5 mg, 1 mg and 2 mg per tablet were prepared as follows:

A) 0.5 mg Tablet:

An 18.00 kg batch of material to be tabletted was prepared as follows:

A flavor mixture was prepared by placing approximately 10 g of Dextrates, NF hydrated (EMDEX) in a plastic bag followed by 42 g of peppermint oil and the contents of the bag were mixed for about 5 minutes then screened using a Comil equipped with a 30 mesh screen with no spacer. To the screened material, approximately 50 g of Dextrates, NF hydrated (EMDEX) and 27 grams of FD&C Red #40 HT Aluminium Lake were added and mixed for about 5 minutes. Following the mixing, 113 g of colloidal silicon dioxide, NF (CAB-O-SIL M-5) was added to the dyed flavor mixture and mixed for an additional 2 minutes.

The following materials were screened using a Comil equipped with a 30 mesh stainless steel screen with no spacer:

7,752.0 g of Mannitol (PEARLITOL SD-100)

the flavor mixture prepared above

600 g of L-Tartaric Acid

600 g of Aspartame, NF

900 g of Povidone USP (KOLLIDON K-30)

1350 g of Crospovidone, NF (POLYPLASSDONE XL-10)

4,922 g of Dextrates, NF Hydrates (EMDEX)

The above screened material was charged into a 2 cu. Ft. blender with 1,545 g of the risperidone granules prepared in Stage I. The amount of risperidone granules was adjusted based upon the actual assay value of the granules which was 97.1%. Accordingly, the amount of mannitol was adjusted based upon the actual weight of risperidone granules used by the following formula: 9.297 kg-(actual weight of risperidone granules) =amount of mannitol. The materials were blended for approximately 20 minutes then screened and blended for an additional twenty minutes after which 90 g of screened magnesium stearate, NF was added to the blender and blended for an additional five minutes.

The final blended material was then compressed into approximately 150,000 tablets using a HealthStar high speed press with the following conditions:

| | |
|---|---|
| Punch: | 0.3125 Round Shape |
| Individual Weight: | 120 mg (110.4 mg-129.6 mg) |
| Hardness: | 1.7 kp (0.7-2.7 kp) |
| Thickness: | 0.115-0.135 inches |

The resulting tablets were tested for disintegration using the procedure <701> described in USP 25 without a disk and 1000 ml low form beaker, purified water at 37±2° C.

In the first trial all six tablets disintegrated within 17 seconds, the second trial all six tablets disintegrated within 15 seconds and the third trial all six tablets disintegrated within 16 seconds.

B) 1.0 mg Tablet

1.0 mg tablets were prepared according to the procedure described in Stage II, part A above except no dye was added to the flavor mixture was prepared. The batch had the following composition:

| | |
|---|---|
| Risperidone Granules | 3.090 kg |
| Tartaric Acid, NF | 0.450 kg |
| Povidone USP (Kollidon K-30) | 0.675 kg |
| Aspartame, NF | 0.450 kg |
| Peppermint Oil, NF | 0.030 kg |
| Crospovidone, NF (Polyplassdone XL-10) | 1.320 kg |
| Mannitol (pearlitol SD-100) | 7.659 kg |
| Dextrates*, NF Hydrated (Emdex) | 4.131 kg |
| Colloidal Silicon Dioxide (Cab-O-Sil M-5) | 0.105 kg |
| Magnesium Stearate, NF | 0.090 kg |

*this amount include the 60 g used to make the flavor mixture

The amount of mannitol was adjusted according to the actual amount of assayed granules employed by the following formula:
10.749 kg−(actual weight of risperidone granules).

The resulting tablets were tested for disintegration according to the procedure described above with the following results: all six tablets in the first trial disintegrated within 49 seconds; all six tablets in the second trial disintegrated within 27 seconds; and all six tablets in the third trial disintegrated within 33 seconds.

C) 2.0 mg Tablet

2.0 mg tablets were prepared according to the procedure described above in Stage II, part A. The batch had the following composition:

| | |
|---|---|
| Risperidone Granules | 6.179 kg |
| Tartaric Acid, NF | 0.150 kg |
| Povidone USP (Kollidon K-30) | 0.225 kg |
| Aspartame, NF | 0.150 kg |
| Peppermint Oil, NF | 0.006 kg |
| Crospovidone, NF (Polyplassdone XL-10) | 1.260 kg |
| Mannitol (pearlitol SD-100) | 7.393 kg |
| Dextrates*, NF Hydrated (Emdex) | 2.430 kg |
| Colloidal Silicon Dioxide (Cab-O-Sil M-5) | 0.090 kg |
| Magnesium Stearate, NF | 0.090 kg |
| D&C Yellow #10 HT Aluminium Lake | 0.027 kg |

*this amount include the 60 g used to make the flavor mixture

The amount of mannitol was adjusted according to the actual amount of assayed granules employed by the following determined according to the following formula:
13.572 kg−(actual weight of risperidone granules).

The resulting tablets were tested for disintegration according to the procedure described above with the following results: all six tablets in the first trial disintegrated within 45 seconds; all six tablets in the second trial disintegrated within 42 seconds; and all six tablets in the third trial disintegrated within 39 seconds.

### EXAMPLE 2

An antidepression tablet containing 15 mg of mirtazapine is prepared by first preparing a drug granulation then blending the granules with tablet excipients and compressing the blend into a tablet.

The granulation should have the following composition:

| | |
|---|---|
| Mirtazapine | 15.0 mg/unit |
| Dextrates, NF Hydrated (EMDEX) | 33.0 mg/unit |
| Croscarmellose Sodium, NF (Ac-Di-Sol) | 0.5 mg/unit |
| Aspartame, NF | 1.0 mg/unit |
| Povidone USP (Kollidon K-30) | 0.375 mg/unit |
| Colloidal Silicon Dioxide, NF (Cab-O-Sil) | 0.125 mg/unit |

The granules are prepared by dissolving the povidone in purified water. The miratazapine, dextrates, croscarmellose sodium and aspartame are screened then placed in a high shear granulator and granulated with the povidone solution. The granules are dried in a drier then passed through a mill and mixed with the colloidal silicon dioxide. After the dried granules are mixed with the colloidal silicon dioxide, they are then mixed with the following excipients in a blender:

| | |
|---|---|
| PHARMBURST* B1 | 241 mg/unit |
| Croscarmellose sodium, NF (Ac-Di-Sol) | 3.5 mg/unit |
| Colloidal Silicon Dioxide, NF (Cab-O-Sil) | 1.3 mg/unit |
| Artificial Grape Flavor | 6.2 mg/unit |
| Magnesium Stearate, NF | 8.0 mg/unit |

*PHARMABURST is a commercially available product from SPI Pharma, Inc. which is a proprietary blend of starch and polyols.

Once the excipients are blended with the granulate, they are compressed into tablet using a high speed press. The target hardness for the tablets is 2 to 5 kp with the preferred hardness being 3.5 kp.

### EXAMPLE 3

An antidepression tablet containing 15 mg of mirtazapine is prepared by first preparing a drug granulation then blending the granules with tablet excipients and compressing the blend into a tablet.

The granulation should have the following composition:

| | |
|---|---|
| Mirtazapine | 48% |
| Dextrates, NF Hydrated (EMDEX) | 48% |
| Croscarmellose Sodium, NF (Ac-Di-Sol) | 1% |
| Aspartame, NF | 2.0% |
| Povidone USP (Kollidon K-30) | 0.75% |
| Colloidal Silicon Dioxide, NF (Cab-O-Sil) | 0.25% |

The granules are prepared by dissolving the povidone in purified water. The miratazapine, dextrates, croscarmellose sodium and aspartame are screened then placed in a high shear granulator and granulated with the povidone solution. The granules are dried in a drier then passed through a mill and mixed with the colloidal silicon dioxide. After the dried granules are mixed with the colloidal silicon dioxide, they are then mixed with the following excipients in a blender:

| | |
|---|---|
| Mirtazapine Granules | 10.08% |
| PHARMBURST* B1 | 84.03% |
| Croscarmellose sodium, NF (Ac-Di-Sol) | 1.0% |
| Colloidal Silicon Dioxide, NF (Cab-O-Sil) | 0.39% |
| Artificial Grape Flavor | 2.0% |
| Magnesium Stearate, NF | 2.5% |

Once the excipients are blended with the granulate, they are compressed into tablet using a high speed press. The target hardness for the tablets is 2 to 5 kp with the preferred hardness being 3.5 kp.

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications thereof which do not depart from the spirit and scope of the invention.

### EXAMPLE 4

5 mg, 7.5 mg, 10 mg, 15 mg and 20 mg olanzapine containing tablets may be prepared according to the procedure outlined in examples 1 or 2 above.

### EXAMPLE 5

A tablet containing 10 mg of carbidopa and 100 mg of levodopa may be prepared according to the procedure outlined in examples 1 or 2 above.

1. A rapidly disintegrating oral pharmaceutical dosage formulation comprising: a water insoluble or slightly water soluble neurological agent wherein the total weight of the tablet is less than 500 mg.

2. The rapidly disintegrating pharmaceutical dosage formulation as defined in claim 1 wherein the total weight of the tablet is less than 400 mg.

3. The rapidly disintegrating pharmaceutical dosage formulation as defined in claim 1 wherein the total weight of the tablet is less than 300 mg.

4. The rapidly disintegrating pharmaceutical dosage formulation as defined in claim 1 wherein the neurological agent is an antipsychotic agent.

5. The rapidly disintegrating pharmaceutical dosage formulation as defined in claim 4 wherein the antipsychotic agent is olanzapine.

6. The rapidly disintegrating pharmaceutical dosage formulation as defined in claim 4 wherein the antipsychotic agent is risperdione.

7. The rapidly disintegrating pharmaceutical dosage formulation as defined in claim 1 wherein the neurological agent is an antidepressant agent.

8. The rapidly disintegrating pharmaceutical dosage formulation as defined in claim 7 wherein the antidepressant agent is mirtazapine.

9. The rapidly disintegrating pharmaceutical dosage formulation as defined in claim 1 further comprising a filler, a binder, a taste enhancing agent, a disintegrant and optionally a stabilizer.

10. The rapidly disintegrating pharmaceutical dosage formulation as defined in claim 1 comprising (a) granules and (b) tableting excipients wherein the granules comprise the psychopharmacological agent, a filler, a binder, a taste enhancing agent, a disintegrants and optionally a stabilizer; and (b) tableting excipients comprise a filler, a disintegrant and a taste enhancing agent.

11. A method for preparing a rapidly disintegrating pharmaceutical dosage formulation comprising the steps of: (a) preparing granules that comprise a water insoluble or slightly water soluble neurological agent, a filler, a binder, a disintegrant, a taste enhancing agent and optionally a stabilizer; (b) blending the granules prepared in step (a) with tableting excipients; and (c) compressing the blend of step (b) into a tablet.

12. The method recited in claim 11 wherein the tableting excipients are selected from the group consisting of a filler, a taste enhancing agent, a disintegrant and a stabilizer.

13. The method of claim 11 wherein the neurological agent is an antipsychotic agent.

14. The method of claim 13 wherein the antipsychotic agent is olanzapine.

15. The method of claim 13 wherein the antipsychotic agent is risperdione.

16. The method of claim 11 wherein the neurological agent is an antidepressant agent.

17. The method of claim 16 wherein the antidepressant agent is mirtazapine.
